(19) United States
(12) Patent Application Publication
(10) Pub. No.: US 2018/0236512 A1
(43) Pub. Date: Aug. 23, 2018

(54) DISPOSAL SYSTEM OF MIXED WASTE OF BURIED LIVESTOCK CARCASS AND CONTAMINATED SOIL AND DISPOSAL METHOD USING THE SAME

(71) Applicant: Hannam University Industry Academy Cooperation Foundation, Daejeon (KR)

(72) Inventors: Geon Ha KIM, Seoul (KR); Young Jun Kim, Daejeon (KR)

(21) Appl. No.: 15/841,405

(22) Filed: Dec. 14, 2017

(30) Foreign Application Priority Data
Feb. 20, 2017 (KR) 10-2017-0022209

(51) Int. Cl. B09B 3/00; C10B 53/07; C12M 1/33; C05F 1/00; C05F 1/02; C05D 9/00; C10L 1/02

(52) U.S. Cl. CPC B09B 3/0083; C10B 53/07; C12M 45/02; C05F 1/005; C05F 1/02; C10L 2290/02; C10L 1/02; C10L 2290/26; C10L 2290/28; C10L 2200/0476; C05D 9/00

(57) ABSTRACT

The present invention relates to a disposal system of a mixed waste of a livestock carcass and a contaminated soil from a burial site of a livestock carcass which is killed and then buried due to infectious livestock diseases such as foot-and-mouth disease or avian influenza, and a disposal method using the same.

### CROSS-REFERENCE TO RELATED APPLICATIONS

This application claims the priority of the Korean Patent Application NO 10-2017-0022209 filed on Feb. 20, 2017 in the Korean Intellectual Property Office, the disclosure of which is incorporated herein by reference.

### BACKGROUND OF THE INVENTION

### Field of the Invention

The present invention relates to a disposal system of a mixed waste of a livestock carcass and a contaminated soil from a burial site of a livestock carcass which is killed and then buried due to infectious livestock diseases such as foot-and-mouth disease or avian influenza, and a disposal method using the same.

### Description of the Related Art

The livestock carcass died by foreign animal diseases such as a foot-and-mouth disease, an avian influenza, and a hog cholera etc. or the livestock carcass stamped out by the infection or infection doubt are treated through a burying method or a thermophilic aerobic method etc.

Most livestock carcass is buried in Korea so as to quickly treat them at a low cost. The area of burying the livestock carcass is called a livestock burial site.

In the livestock burial site, it prohibits the excavation and utilization thereof for a certain period of time in accordance with Domestic Animal Infectious Disease Control Law and follow-up management guideline. However, when the specified time has elapsed, the livestock burial site is available as the original land object. Thus, after considerable space of time, it cultivates crops in the livestock burial site. At this time, if it digs in the ground of the livestock burial site, there is a common occurrence that the livestock carcass is not decayed to be remained. The agricultural products produced in the livestock burial site having non-biodegradable livestock carcass or in the surrounding thereof damage the image of local agricultural products. Also, since the shallow ground water as a potential pollution source can be discharged from the livestock burial site in the low water season and the pollution source consistently flows into the stream during rainfall, it can pollute the neighboring river and cause damage to the soil owing to the odor and the remaining antibiotic.

In order to minimize these damages, it requires the follow-up management of the livestock burial site. Moreover, the research and development on the treating method for effectively destroying the livestock burial site has been required.

### PATENT LITERATURE

Patent Literature 1: Korean Patent Registration No. 10-1328070 (Nov. 15, 2013)

### SUMMARY OF THE INVENTION

The present invention has been made to solve the problems occurring in the prior art, and an object thereof is to provide a disposal system of a mixed waste of a buried livestock carcass and a contaminated soil which efficiently performs a destruction treatment of a livestock burial site and a disposal method using the same.

Specifically, the present invention has been made in an effort to further provide a disposal system of a mixed waste of a buried livestock carcass and a contaminated soil which may quickly treat a mixed waste according to a state of a livestock burial site and maximize a treatment performance and a disposal method using the same.

Further, another object of the present invention is to provide a disposal system of a mixed waste of a buried livestock carcass and a contaminated soil which is capable of utilizing an energy source generated during a treatment process for a linked process to supply energy by itself during the entire process to maximize energy efficiency and a disposal method using the same.

Furthermore, another object of the present invention is to provide a disposal system of a mixed waste of a buried livestock carcass and a contaminated soil which utilizes recyclable by-products which may be obtained during the treatment process to consistently operate the system for a long time and produces a functional material such as a biochar to create an added value and a disposal method using the same.

Moreover, another object of the present invention is to provide a disposal system of a mixed waste of a buried livestock carcass and a contaminated soil which obtains stabilized by-products which can be treated in a treatment facility of the related art by treating the mixed waste of the buried livestock carcass and the contaminated soil, and safely transports the by-product to the treatment facility of the related art to perform excreta treatment, special burial, incineration, and agricultural land application etc. and a disposal method using the same.

According to an aspect of the invention to achieve the object described above, there is provided a disposal system of a mixed waste of a buried livestock carcass and a contaminated soil, including: a pre-treatment tank which separates and shreds a waste; a treatment tank which treats the waste separated in the pre-treatment tank using bio-reaction or plant purification and includes an anaerobic digesting unit, an aerobic digesting unit, and a plant purifying unit; and a post-treatment tank which converts by-products generated in the treatment tank into energy to obtain a biofuel and a biochar.

According to an embodiment of the present invention, the disposal system obtains stabilized by-products which can be treated in a treatment facility of the related art by treating the mixed waste of the incoming buried livestock carcass and contaminated soil, and safely transports the by-product to the treatment facility of the related art to perform excreta treatment, special burial, incineration, and agricultural land application.

According to an embodiment of the present invention, the treatment tank is supplied with biofuel obtained in the post-treatment tank of a subsequent stage to use the biofuel as an energy source.

According to another aspect of the invention to achieve the object described above, there is provided a disposal method of a mixed waste of a buried livestock carcass and a contaminated soil, including: (a) a pre-treatment step of separating and shredding a waste of a livestock burial site; (b) a treatment step of treating the waste separated in the pre-treatment step using one or more processes selected from an anaerobic digestion process, an aerobic digestion process, and a plant purification process; (c) a post-treatment step of converting by-products generated in the treatment step into energy using a biomass or producing a biochar; and (d) a linkage treatment step of transporting the by-products obtained in the pre-treatment step, the treatment step, or the post-treatment step to perform one or more processes selected from excreta treatment, special burial, incineration, and agricultural land application.

According to an embodiment of the present invention, in the separated waste of the treatment step (b), the livestock carcass from which the contaminated soil is separated is transported to the anaerobic digestion process, the livestock carcass from which the contaminated soil is not separated is transported to the aerobic digestion process, and the contaminated soil is transported to the plant purification process to be treated.

According to an embodiment of the present invention, the plant purification process removes the contaminants having nitrogen, antibiotics, and pathogens in the collected contaminated soil.

According to an embodiment of the present invention, during the plant purification process, the plant is cultivated in a ground where the collected contaminated soil and the biochar are mixed to remove the contaminants in the contaminated soil.

### BRIEF DESCRIPTION OF THE DRAWINGS

The above and other objects, features and advantages of the present invention will be more apparent from the following detailed description taken in conjunction with the accompanying drawings, in which:

FIG. 1 is a schematic block diagram illustrating a disposal system of a mixed waste of a livestock carcass and a contaminated soil according to one embodiment of the present invention;

FIG. 2 is a schematic flow chart illustrating a disposal method of a mixed waste of a livestock carcass and a contaminated soil according to one embodiment of the present invention; and

FIG. 3 is a schematic diagram illustrating a linked combination process in which a biomass generated by a treatment step of the present invention is changed into energy in a post-treatment step and it is again supplied to the treatment step.

### DETAILED DESCRIPTION OF THE PREFERRED EMBODIMENT

Hereinafter, preferred embodiments of the invention will be described in more detail with reference to the accompanying drawings.

In the present invention, a “livestock burial site” refers to a ground where carcasses of livestock which is infected with livestock infectious diseases or has an epidemiological investigation or detailed investigation result or a clinical sign of infectious diseases are buried.

In the present invention, unless specifically mentioned, a “waste” refers to a mixed waste of a livestock carcasses buried in a livestock burial state and a soil contaminated from the livestock carcasses.

In the present invention, a “contaminated soil” refers to a contaminated soil including contaminants such as antibiotics or pathogenic bacteria due to the carcasses of the livestock.

In the present invention, “dead livestock” refers to a carcass of livestock and is understood as the same meaning as a carcass.

The disposal system of a mixed waste of a buried livestock carcass and a contaminated soil and the disposal method according to the present invention may be performed by sequentially or organically linking a pre-treatment process, a treatment process, and a post-treatment process. Simultaneously, without adding a separate process or changing the processes, the disposal system and the disposal method may be combined with a step processed by one or more processes selected from an anaerobic digestion process, an aerobic digestion process, and a plant purification process according to various conditions such as a state of a waste separated during the pre-treatment process, that is, a state of a livestock burial site, a type and a shape of a dead livestock in the livestock burial site, to immediately correspond. Therefore, it is possible to shorten the treatment time by means of a simple process and maximize the efficiency of livestock burial site destruction process. Particularly, a linked combination of the treatment process and the post-treatment process allows the by-products generated during the treatment step to be recycled as a biomass so that energy efficiency may be increased and stabilization of the waste is accelerated. Therefore, it is very advantageous for a linkage treatment.

An aspect of a disposal system of a mixed waste of a buried livestock carcass and a contaminated soil includes a pre-treatment tank which separates and shreds a waste, a treatment tank which treats the waste separated in the pre-treatment tank using bio-reaction or plant purification and includes an anaerobic digesting unit, an aerobic digesting unit, and a plant purifying unit, and a post-treatment tank which converts by-products generated in the treatment tank into an energy to obtain a biofuel and a biochar.

The pre-treatment tank separates and shreds the waste supplied from the livestock burial site to increase an active surface area of the waste. Therefore, the pre-treatment tank includes a hopper to facilitate insertion of the waste. In this case, since the contaminated soil is mixed in the supplied waste, in order to reduce an amount of the contaminated soil, the waste may be transported through a vibration conveyer belt before being inserted into the hopper. In addition, the pre-treatment tank may further include a screen unit to filter a lumped dead livestock waste and soil from the shredded waste. Further, the pre-treatment tank may further include a compression molding machine which compressively molds the waste to facilitate shredding of the waste, before inserting the waste into the hopper. A liquid waste which is generated due to the compression is separated and collected through a separate leaching pipe to be treated.

The treatment tank performs the bio-reaction or plant purification on the waste separated in the pre-treatment tank and includes any one or more selected from an anaerobic digesting unit, an aerobic digesting unit, and a plant purifying unit.

The anaerobic digesting unit and the aerobic digesting unit reduce a volume of the waste through the bio-reaction or stabilize the waste for a subsequent linkage treatment.

The anaerobic digesting unit includes a thermal hydrolysis reactor which is capable of performing high temperature and high pressure treatment. Further, the anaerobic digesting unit may further include a high pressure rendering device. The aerobic digesting unit includes a moisture supplying unit and a stirring unit. More desirably, the anaerobic digesting unit and the aerobic digesting unit may further include means of a thermal treatment unit or microorganism to increase process efficiency.

The plant purifying unit purifies the contaminated soil using plants cultivated in the soil and includes a ground where the contaminated soil and the biochar are mixed and the plant cultivated in the ground.

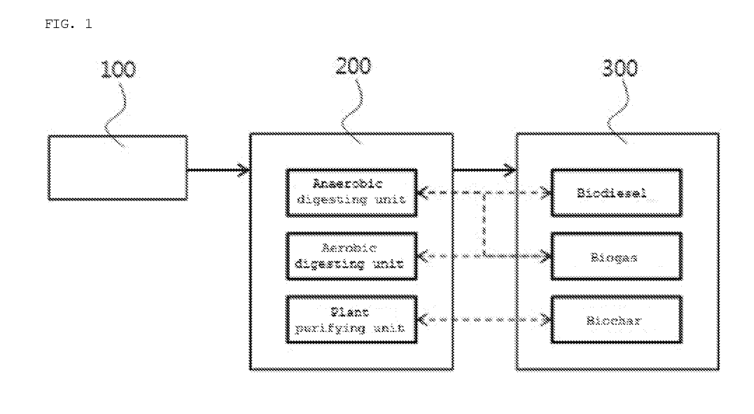

The disposal system according to the present invention may unify the entire process by organically linking the pre-treatment tank (100), the treatment tank (200), and the post-treatment tank (300), as illustrated in FIG. 1. Particularly, the treatment tank may be supplied with biofuel such as biodiesel or biogas obtained in the post-treatment tank of a subsequent stage to use the biofuel as an energy source. By doing this, the biomass obtained from the by-products generated during the process is recycled as an energy source and thus the biofuel such as the biodiesel or biogas, which is generated in the post-treatment tank, is used again in the treatment tank. Therefore, the energy efficiency may be maximized.

Further, the biofuel and the biochar produced in the post-treatment tank may be supplied to farms and a part of the biofuel and the biochar is utilized in the linkage treatment step, so that the cost may be saved.

The by-products obtained through the disposal system is stabilized to be treated in the treatment facility (an excreta treatment facility, an incineration plant, a special burial site, etc.) of the related art and is safely transported to a treatment facility of the related art to perform excreta treatment, special burial, incineration, and agricultural land application.

An aspect of the disposal method of the mixed waste of the buried livestock carcass and the contaminated soil according to the present invention includes: (a) a pre-treatment step of separating and shredding a waste of a livestock burial site, (b) a treatment step of treating the waste separated in the pre-treatment step using one or more processes selected from an anaerobic digestion process, an aerobic digestion process, and a plant purification process, (c) a post-treatment step of converting by-products generated in the treatment step into energy using a biomass or producing a biochar, and (d) a linkage treatment step of transporting the by-products obtained in the pre-treatment step, the treatment step, or the post-treatment step to perform one or more processes selected from excreta treatment, special burial, incineration, and agricultural land application.

In the present invention, the livestock burial site is a burial site which is vulnerable in terms of a safety. Therefore, in order to prevent persistent groundwater contamination due to potential contamination source and safely cultivate crops, soil and carcasses in the waste of the burial site which is pre-treated and post-treated need to be separated. According to the above-described treatment, the present invention may restore the soil from the waste of the livestock burial site to a farm land and the dead livestock is stabilized through the anaerobic or aerobic digestion (or decomposition) process according to a state of the dead livestock and then linkage treatment step is performed to apply the dead livestock to a special burial site, a excreta treatment facility, an incineration plant, or an agricultural land.

In the present invention, the pre-treatment step (a) is a process for increasing an active surface area of the waste and filtering the soil which is lumped with the carcass as much as possible to increase the efficiency of the entire burial site destruction process.

In the pre-treatment step, a process of separating and shredding the waste excavated from the livestock burial site is performed. In this case, a shredder is used to shred the waste. If a shredder shreds the carcass into a uniform size, the shredder may be used without any limitation. Desirably, an appropriate interval is set so that a case where a skin or internal organs of the carcass are not shredded and droop does not occur. For example, the shredder may be configured such that a plurality of rotary shafts equipped with blades therein is interlocked with each other so that the carcass passes through the rotating blades are shredded. A size of the shredded carcass is not specifically limited as long as it is within a range to achieve the object of the present invention. However, desirably, the size of the shredded carcass may be 10 cm or smaller, that is, within a range of 1 to 10 cm.

In the shredding process, the waste is transported for the shredding process. That is, the waste may be transported to the shredder through a conveyer belt after being excavated from the livestock burial site. It is preferable to use a vibration conveyer belt as the conveyer belt, since the soil on the carcass during the transporting process may be removed by vibration.

Further, the transported waste is compressively molded using a molding cast before inserting the transported waste into the shredder through a hopper and then the molded waste is inserted into the shredder. The compression molding process is a process of applying a pressure to the waste to be molded so that carcass shredding performance and efficiency may be increased. Since pressure is applied during the compression molding process, a liquid waste which is separated from a solid material is generated from the waste. This may be separately collected using a leaching pipe to be treated. The liquid waste is sent to a fat-and-oil factory to be recycled as fertilizer or industrial fats and oils. The solid material which is a molded waste may be subjected to a cooling process. The cooling process may be performed using sub-zero wind after a sprinkling process, but is not limited thereto. By doing this, hard-to-shred parts such as skins or internal organs may be smoothly shredded.

The pre-treatment step (a) may further include a vibration screening process after the shredding. During the vibration screening process, the shredded waste is separated into a dead livestock waste and a contaminated soil so that the contaminated soil may be effectively filtered. In this case, the size of the screen may be adjusted within a range to achieve the object of the present invention.

The carcass on which the pre-treatment is completed through the pre-treatment step (a) is linked to the treatment step (b) which is a subsequent process. This may include a process of transporting a predetermined amount after being stored in a yard in the facility to be applied to the treatment step.

In the present invention, the treatment step (b) is a process of treating the waste separated in the pre-treatment step using one or more processes selected from the anaerobic digestion process, the aerobic digestion process, and the plant purification process.

The treatment step (b) includes a process of transporting the pre-treated waste after classifying a state of the waste according to specific criteria to insert the pre-treated waste to the above-described process. In this case, the state of the waste is determined depending on whether the contaminated soil is separated therefrom. The carcass from which the contaminated soil is separated is transported to the anaerobic digestion process and the carcass from which the contaminated soil is not separated is transported to the aerobic digestion process. Further, the contaminated soil is transported to the plant purification process.

As the state of the waste, whether the contaminated soil is separated from the carcass is determined based on a content of the contaminated soil of the pre-treated waste or the contaminated soil adhered on an outer surface of the carcass. For example, the classification may be determined by setting a reference of the content of the contaminated soil or measuring an apparent area of the contaminated soil attached on the carcass.

The anaerobic digestion process is a process of liquefying the carcass which is a solid waste so as not to leave residues. According to the anaerobic digestion process, a carcass which maintains an original shape or is insufficiently decomposed is shredded first and the shredded carcass and the leachate of the burial site are treated in a thermal hydrolysis reactor at a high temperature and a high pressure. In this case, anaerobic bacteria may be utilized as a method using microorganisms.

The anaerobic digestion process may include a high pressure rendering process as a previous or subsequent step. The rendering process is sterilization with a high temperature and high pressure steam and removes the virus so that the possibility of secondary contamination is blocked.

In the present invention, a thermal hydrolysis reactor may be used for the anaerobic digestion process and the anaerobic digestion process may be better if the carcass and the contaminated soil are not mixed. For example, it is more effective for the dead livestock which is separately disposed in a fiber reinforced plastic (FRP) storage tank in the livestock burial site.

Further, during the anaerobic digestion process, floating oil of final by-products after performing the anaerobic digestion process may be used as a biomass. The biomass may be applied to produce a biochar during the plant purification process of the contaminated soil and also utilized as an energy source such as a biodiesel so as to be reused for the process.

The aerobic digestion process is intended to reduce a volume of the waste and increase reduction efficiency through decomposition and may be performed by supplying water and stirring. Specifically, the aerobic digestion process may be performed through fermentation process after adding and stirring additives containing aerobic microorganism while supplying water like a sprinkling process.

The fermentation process may be performed while standing still and may further include a process of inducing cell activation of aerobic microorganism. For example, a high temperature status in the range of 60 to 80° C. may be maintained. Thereafter, the by-products are aged to compost the waste. The production of composts using the dead livestock as a raw material is prohibited by the relevant laws. However, in the present invention, the waste may be utilized by being supplied to the soil cultivated during the plant purification process.

Further, biogas may be extracted using methane gas produced during the anaerobic digestion process. The biogas may be recycled as a thermal energy source in the treatment step of the present invention. Therefore, it is advantageous because the energy source may be supplied by itself without receiving a separate energy source from the outside.

During the aerobic digestion process, the water is consistently supplied and stirred so that it is advantageous to treat the carcass which is not separated from the contaminated soil. Therefore, as described above, when the classification is performed based on the state of the waste separated during the pre-treatment step, the carcass in this state is applied to the aerobic digestion process. When the aerobic digestion process is not performed, it is better to perform the anaerobic digestion process which is desirable in teams of the treatment time and a residue treatment. According to the present invention, the processes are promptly applied according to the state of the waste to perform a linked process. Therefore, the process efficiency may be increased.

Further, in the present invention, the above-described anaerobic digestion process and aerobic digestion process are processes of accelerating stabilization of the waste and include a process of transporting the stabilized waste to a subsequent process, including exporting the waste to the outside.

It is difficult to directly apply the contaminated soil separated through the pre-treatment step (a) to the agricultural land because the contaminated soil contains pieces of carcass, vinyls or gravels. Particularly, a nitrogen component of the contaminated soil may be transferred to crops and the contaminated soil contains contaminants which are trace-organic matters such as antibiotics and pathogens. The nitrogen of the contaminated soil may be reduced and the contaminants such as antibiotics and pathogens may be removed from the contaminated soil through the plant purification process.

In the present invention, the plant purification process is a process of removing or stabilizing harmful contaminants in the contaminated soil by using plants.

During the plant purification process, the plant is cultivated in a ground where the collected contaminated soil and the biochar are mixed to remove the contaminants in the contaminated soil.

In this case, the biochar is produced by thermally decomposing the biomass. Desirably, the biochar may be produced by receiving the biomass generated during the previous anaerobic digestion process. Therefore, the anaerobic digestion process and the plant purification process are linked so that the disposal system of the present invention may be unified.

The biochar may be produced in the post-treatment step which is a subsequent process of the treatment step. As a specific example, the biomass supplied from the anaerobic digestion process is accommodated in a closed container and is indirectly heated under an oxygen-free atmosphere to thermally decompose the biomass so that the biomass may be transformed to the biochar. In this case, the oxygen-free atmosphere may be a state where no oxygen is provided. However, the heating may be performed at a low-oxygen atmosphere under a premise that ignition by oxygen is minimized or hardly generated. The indirect heating does not mean that the biomass is directly heated, but that the container itself is heated or the biomass accommodated in the container is heated through indirect heating such as high frequency heating. A thermal decomposition temperature of the biomass according to the heating may be adjusted within a range of producing the biochar, and may desirably be 400 to 900° C. When the thermal decomposition temperature is lower than the above-mentioned temperature range, oil contained in the biomass is discharged to the outside so that the thermal decomposition is not smoothly performed. In contrast, when the thermal decomposition temperature exceeds the above-mentioned temperature range, the biomass may be carbonized before being thermally decomposed.

The produced biochar has a high surface area and microporous structure. When the biochar is mixed with the contaminated soil, it is effective in adsorbing trace-organic matters contained in the contaminated soil and may move the contaminated material adsorbed on the biochar to the plant according to the growth of the cultivated plant during the plant purification process. The plant is harvested and then applied in production of the biochar so that it is advantageous to ensure a circulation structure of a system which efficiently removes the contaminated soil.

The biochar may have a bead shape, but is not limited thereto. Further, as another aspect, the biochar may be utilized to construct a barrier wall which prevents the leachate generated during the pre-treatment step or the treatment step from leaking to the ground water. That is, the plant purification process of the present invention may be linked with the aerobic digestion process and the anaerobic digestion process to utilize the produced biochar. Further, the plant purification process may accomplish the semi-permanent purification by adsorbing the contaminated materials in the contaminated soil using the biochar and moving the contaminants into the cultivated plant by the self-organizing power of the cultivated plant.

The soil purified by the plant purification process may be mixed with a good quality soil or the biochar to be applied to the agricultural land or applied for beautification around the destruction burial site.

In the present invention, the post-treatment step (c) is a process of producing a biofuel such as a biodiesel or biogas or the biochar using a biomass which is an organic by-product produced in one or more processes selected from the anaerobic digestion process, the aerobic digestion process, and the plant purification process. This regenerates the by-product generated during the treatment step as a biomass. Therefore, it is advantageous since the energy source is supplied by itself according to the disposal method according to the present invention and the treatment process is unified.

The biodiesel, the biogas, and the biochar generated in the post-treatment step may be used as an energy source in the above-described treatment step.

Further, the biofuel and the biochar may be supplied to a farm or utilized in the linkage treatment step so that the cost may be saved.

The linkage treatment step (d) is a step of transporting the by-products obtained in the pre-treatment step, the treatment step or the post-treatment step to the treatment facility of the related art. The obtained by-product is stabilized to be treated in the treatment facility (an excreta treatment facility, an incineration plant, or a special burial site, etc.) of the related art and is safely transported to the existing treatment facility to perform an excreta treatment, incineration, special burial, or agricultural land application.

For example, the leachate generated during the pre-treatment step or the treatment step is transported to the livestock excreta and sanitary excreta treatment facility to be linked and may also be linked to the wastewater treatment facility or incineration plant to be treated. In this case, when a pH treatment is performed on the leachate and then the linkage treatment is performed, the leachate is transported by adjusting a contaminated load amount of a total nitrogen or total phosphorus to have a load within a range which does not interrupt a normal operation. Further, the by-products generated in the anaerobic digestion process or the aerobic digestion process are incinerated and a linkage treatment which applies the soil treated during the plant purification process to the agricultural land may be performed.

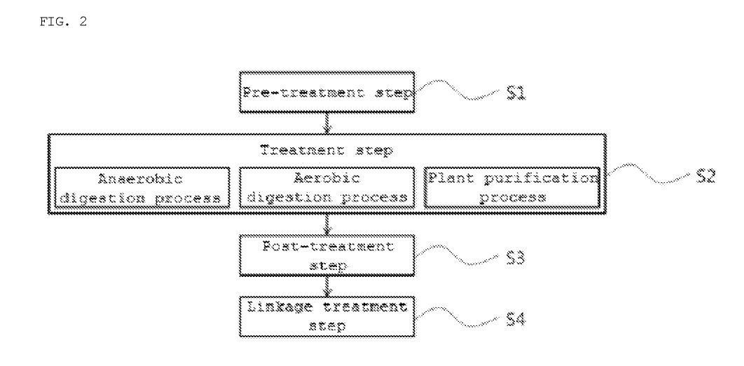

According to an aspect of the disposal method of the mixed waste of the buried livestock carcass and the contaminated soil according to the present invention, as illustrated in FIG. 2, the pre-treatment step (S1), the treatment step (S2), the post-treatment step (S3), and the linkage treatment step (S4) are sequentially performed. The by-products generated in the steps are linked to each other to unify and combine the entire processes as one process. Therefore, the mixed waste may be quickly and economically treated. The by-product obtained through the disposal system is stabilized to be treated in the treatment facility (an excreta treatment facility, an incineration plant, a special burial site, etc.) of the related art and is transported to a treatment facility of the related art through the linkage treatment step (S4) to perform excreta treatment, special burial, incineration, and agricultural land application etc.

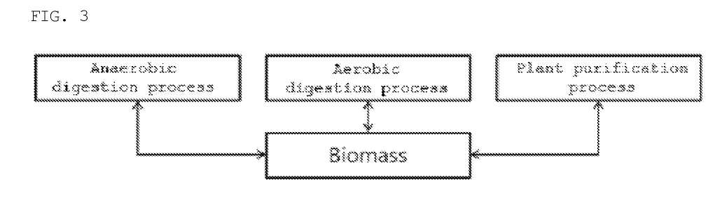

Further, as illustrated in FIG. 3, the biomass obtained from the by-products generated in the treatment step is recycled so that the biofuel such as a biodiesel or biogas or biochar are converted into energy and is supplied to any one or more processes selected from the anaerobic digestion process, the aerobic digestion process, and the plant purification process of the treatment step to be used as an energy source. Therefore, the energy efficiency of the entire processes may be maximized and the waste may be consistently stabilized for a long time.

According to the present invention, a disposal system of a mixed waste of a buried livestock carcass and a contaminated soil and a method using the same are very efficient in terms of time and cost for destroying a livestock burial site and effectively prevent damages due to odor, land usage, or a potential groundwater contamination near the burial site.

Further, a biomass obtained from the by-products generated during the process is utilized as an energy source so that energy is supplied by itself during the entire process. Thus, the energy efficiency is significantly improved. Therefore, it is advantageous to minimize a supply of an energy source required for the process from a separate external device and unify the entire process by organically linking individual processes.

Further, the destruction process of the livestock burial site is quickly and economically performed so as to significantly reduce an environmental load, the system may be stably operated for a long time. Further, the biomass and the biochar are recycled to create an added value.

Further, the present invention may obtain stabilized by-products which can be treated in a treatment facility of the related art by treating the mixed waste of the buried livestock carcass and the contaminated soil, and safely transport the by-product to the treatment facility of the related art to perform excreta treatment, special burial, incineration, and agricultural land application.

While the present invention has been described with respect to the specific embodiments, it will be apparent to those skilled in the art that various changes and modifications may be made without departing from the spirit and scope of the invention as defined in the following claims.

What is claimed is:

1. A disposal system of a mixed waste of a buried livestock carcass and a contaminated soil, comprising:
a pre-treatment tank which separates and shreds a waste;
a treatment tank which treats the waste separated in the pre-treatment tank using bio-reaction or plant purification and includes an anaerobic digesting unit, an aerobic digesting unit, and a plant purifying unit; and
a post-treatment tank which converts by-products generated in the treatment tank into energy to obtain a biofuel and a biochar.

2. The disposal system of a mixed waste of a buried livestock carcass and a contaminated soil as claimed in claim 1, wherein the disposal system obtains stabilized by-products which can be treated in a treatment facility of the related art by treating the mixed waste of the incoming buried livestock carcass and contaminated soil, and safely transports the by-product to the treatment facility of the related art to perform excreta treatment, special burial, incineration, and agricultural land application.

3. The disposal system of a mixed waste of a buried livestock carcass and a contaminated soil as claimed in claim 1, wherein the treatment tank is supplied with biofuel obtained in the post-treatment tank of a subsequent stage to use the biofuel as an energy source.

4. A disposal method of a mixed waste of a buried livestock carcass and a contaminated soil, comprising:
(a) a pre-treatment step of separating and shredding a waste of a livestock burial site;
(b) a treatment step of treating the waste separated in the pre-treatment step using one or more processes selected from an anaerobic digestion process, an aerobic digestion process, and a plant purification process;
(c) a post-treatment step of converting by-products generated in the treatment step into energy using a biomass or producing a biochar; and
(d) a linkage treatment step of transporting the by-products obtained in the pre-treatment step, the treatment step, or the post-treatment step to perform one or more processes selected from excreta treatment, special burial, incineration, and agricultural land application.

5. The disposal method of a mixed waste of a buried livestock carcass and a contaminated soil as claimed in claim 4, wherein, in the separated waste of the treatment step (b), the livestock carcass from which the contaminated soil is separated is transported to the anaerobic digestion process, the livestock carcass from which the contaminated soil is not separated is transported to the aerobic digestion process, and the contaminated soil is transported to the plant purification process to be treated.

6. The disposal method of a mixed waste of a buried livestock carcass and a contaminated soil as claimed in claim 4, wherein the plant purification process removes the contaminants having nitrogen, antibiotics, and pathogens in the collected contaminated soil.

7. The disposal method of a mixed waste of a buried livestock carcass and a contaminated soil as claimed in claim 6, wherein, during the plant purification process, the plant is cultivated in a ground where the collected contaminated soil and the biochar are mixed to remove the contaminants in the contaminated soil.
